(19) United States
(12) Patent Application Publication
(10) Pub. No.: US 2025/0093247 A1
(43) Pub. Date: Mar. 20, 2025

(54) ADAPTIVE TIME LIGHT SCATTERING AND ELECTROPHORETIC MOBILITY DATA COLLECTION TECHNIQUES

(71) Applicant: Wyatt Technology, LLC, Goleta, CA (US)

(72) Inventors: Steven P. Trainoff, Santa Barbara, CA (US); Cha Lee, Sacramento, CA (US); Shivakumar Ramini, Newbury Park, CA (US)

(21) Appl. No.: 18/889,553

(22) Filed: Sep. 19, 2024

Related U.S. Application Data
(60) Provisional application No. 63/583,605, filed on Sep. 19, 2023.

(51) Int. Cl. G01N 15/0205; G01N 15/00; G01N 15/02

(52) U.S. Cl. CPC G01N 15/0211; G01N 15/0266; G01N 2015/0003; G01N 2015/0277

(57) ABSTRACT

The present disclosure describes a method, system, and computer program product comprising a process whereby measurement data collected by an instrument is collected adaptively until a desired relative or absolute accuracy is achieved.

### RELATED APPLICATIONS

The present application claims priority to U.S. Provisional Patent Application No. 63/583,605, filed Sep. 19, 2023 and entitled “ADAPTIVE TIME LIGHT SCATTERING AND ELECTROPHORETIC MOBILITY DATA COLLECTION TECHNIQUES” the entirety of which is incorporated by reference herein.

The present application is related to U.S. Provisional Patent No. 63/466,211 filed May 12, 2023 and entitled “APPARATUS TO MEASURE ELECTROPHORETIC MOBILITY”, U.S. Provisional Patent No. 63/466,243 filed May 12, 2003 and entitled “FLOW CELL FOR ELECTROPHORETIC MOBILITY MEASUREMENT”, U.S. Provisional Patent No. 63/522,573 filed Jun. 22, 2023 and entitled “CIRCUIT TO CONTROL ACOUSTO-OPTIC MODULATORS FOR MEASURING ELECTROPHORETIC MOBILITY”, U.S. Provisional Patent No. 63/522,608 filed Jun. 22, 2023 and entitled “LASER DRIVER CIRCUIT FOR MEASURING ELECTROPHORETIC MOBILITY OF A SAMPLE”, U.S. Provisional Patent No. 63/528,535 filed Jul. 24, 2023 and entitled “CALCULATING ELECTROPHORETIC MOBILITY OF A SAMPLE BY EXTRACTING SPECTRA”, U.S. Provisional Patent No. 63/528,551 filed Jul. 24, 2023 and entitled “CALCULATING ELECTROPHORETIC MOBILITY OF A SAMPLE BY EXTRACTING FREQUENCY SHIFT”, U.S. Provisional Patent No. 63/529,411 filed Jul. 28, 2023 and entitled “APPARATUS TO ISOLATE VIBRATION FOR ELECTROPHORETIC MOBILITY MEASUREMENT,” and U.S. Provisional Patent No. 63/529,415 filed Jul. 28, 2023 and entitled “APPARATUS TO ISOLATE EXTERNAL VIBRATION FOR ELECTROPHORETIC MOBILITY MEASUREMENT,” the contents of each of which are incorporated by reference in their entirety.

### BACKGROUND

The present disclosure relates to chemical and biological sample analysis techniques, and more specifically, to adaptive techniques that adjust the parameters of acquisition data during their collection to automatically achieve a desired measurement accuracy and precision. The algorithm is general and can be applied to any measurement that is constructed from the aggregated data from a series of shorter acquisitions. Here it is described as applied to dynamic light scattering and electrophoretic mobility detection measurements.

### SUMMARY

The present disclosure describes a computer implemented method, a system, and a computer program product of receiving, from a data store by a computer system, a predetermined threshold for determining a measurement result related to a sample processed by an instrument; collecting data regarding the sample from the instrument until a standard error in a mean among an acquisition series of the data is substantially equal to the predetermined threshold; and generating the measurement result of the acquisition series of the data when the standard error is substantially equal to the predetermined threshold.

In another aspect, described are a computer implemented method, a system, and a computer program product comprising programming a special-purpose computer system with a set of collection parameters including a desired statistical accuracy and a maximum number of acquisitions; collecting acquisition data for determining a measurement, the acquisition data collected according to the set of collection parameters; as each subsequence acquisition is collected, computing the standard error of the mean of all acquisitions that have been completed, wherein when a value of a current acquisition crosses a predetermined threshold or the maximum number of acquisitions is reached, the system returns the measurement.

### BRIEF DESCRIPTION OF THE DRAWINGS

FIG. 1 depicts a block diagram of a system at which embodiments of the present inventive concept can be practiced.

FIG. 2 depicts a flowchart in accordance with an embodiment.

FIG. 3 depicts a graph in accordance with an embodiment.

FIG. 4 depicts a graph in accordance with an embodiment.

FIG. 4A depicts a graph in accordance with an embodiment.

FIG. 5 depicts a table of standard deviation values of a plurality of measurements determined according to the computer implemented method of FIG. 2.

FIG. 6 depicts a computer system in accordance with an exemplary embodiment.

### DETAILED DESCRIPTION

In brief overview, embodiments of the present disclosure describe an apparatus and method that dynamically adjust the parameters used for collecting acquisition data regarding an unknown chemical or biological sample when a measurement is performed on the sample. Parameters may include a time to integrate, a maximum number of acquisitions for a desired sample measurement, and a desired statistical accuracy. The method and apparatus dynamically adjusts the number of acquisitions that constitute a measurement for the desired statistical accuracy to be achieved.

Accordingly, in some embodiments, with a fixed set of collection parameters one can compute the statistics of the reported results such as a mean electrophoretic mobility as determined by computing the mobility on each acquisition (e.g., taken in a 1 second time period) and averaging the results of all previously collected acquisitions for a given measurement. Here, a user can program a special-purpose computer system with a desired statistical accuracy threshold and a maximum number of acquisitions. The statistical accuracy can be specified as a threshold applied to the relative (e.g., percentage) of the mean, or as an absolute error threshold (e.g., measured in mobility units or μm cm (V sec)). As each subsequent acquisition is collected, the system computes the relative standard error of the mean of all acquisitions and the absolute standard error that have been completed, which can be used to determine whether the results achieve a desired precision of accuracy. When the relative standard error crosses the relative standard error desired threshold, or the absolute standard error crosses the absolute standard error threshold, or the maximum number of acquisitions is reached, the number of current acquisitions ceases to be collected and the system returns the measurement. The system then resets and starts collecting acquisitions for the next measurement. This process improves the repeatability and reproducibility of measurements taken with either DLS or electrophoretic mobility detection operations. In other embodiments, in a postprocessing variant, the raw acquisition data is collected and the choice of the error thresholds to use or the maximum number of acquisitions to use per measurement can be adjusted after-the-fact.

### Definitions

### Particle

A particle may be a constituent of a liquid sample aliquot. Such particles may be molecules of varying types and sizes, nanoparticles, virus like particles, liposomes, emulsions, bacteria, and colloids. These particles may range in size from sub-nanometer to microns.

### Light Scattering

Light scattering (LS) is a non-invasive technique for characterizing macromolecules and a wide range of particles in solution. One type of light scattering detection frequently used for the characterization of macromolecules is dynamic light scattering.

### Dynamic Light Scattering

Dynamic light scattering is also known as quasi-elastic light scattering (QELS) and photon correlation spectroscopy (PCS). In a DLS experiment, time-dependent fluctuations in the scattered light signal are measured using a fast photodetector. DLS measurements determine the diffusion coefficient of the molecules or particles, which can in turn be used to calculate their hydrodynamic radius.

### Electrophoretic Light Scattering

Electrophoretic light scattering (ELS) is a technique used to measure the electrophoretic mobility of particles in dispersion, or molecules in solution, or the solute's response to an applied electrical field. This mobility is often converted to Zeta potential to enable comparison of materials under different experimental conditions. The fundamental physical principle is that of electrophoresis. A dispersion is introduced into a cell containing two electrodes. An electrical field is applied to the electrodes, and particles or molecules that have a net charge, or more strictly a net zeta potential, will migrate towards the oppositely charged electrode with a velocity. The ratio of this velocity to the applied electric field is the mobility, which can be measured. It can be expressed as a zeta potential by applying theoretical models.

When an electric field is applied to a sample, any charged objects in the sample will be influenced by that field. The extra movement that particles exhibit in response to the electric field is called the electrophoretic mobility. Its units are μm·cm/V·s (micrometer centimeter per Volt second) since it is a velocity [μm/s] per field strength [V/cm]). The electrophoretic mobility is the direct measurement from which the zeta potential can be derived (using either the Smoluchowski/Debye-Hückel approximations or the complete Henry function F(ca) to convert from the mobility to a zeta potential).

Electrophoretic light scattering (ELS) is based on DLS and involves applying an electric field to the sample in order to exert a force on the (charged) particles In order to prevent the accumulation of charged particles onto the electrodes, an alternating field is used, whose direction is switched (e.g., between positive and negative directions) rapidly enough to prevent charge build-up. During the application of a positive electric field, a positively charged sample acquires a positive velocity component, which leads to a positive Doppler frequency shift on the light that is scattered from the sample. During the application of a negative electric field, the sample acquires a velocity component in the opposite direction, which leads to a negative Doppler shift on the light that is scattered from the sample.

### Current Technologies

Experiments in dynamic light scattering and electrophoretic mobility detection are typically conducted by setting a fixed integration time for each sample data acquisition and collecting several acquisitions that are aggregated into a measurement of the same sample. The measurement data may allow various results such as size or electrophoretic mobility to be computed. For example, an electrophoretic mobility measurement may consist of 15 acquisitions with a 1 second integration time for each. A dynamic light scattering (DLS) measurement may consist of 20 acquisitions each of which is integrated for 5 seconds. Even though the fundamental data collected by each analysis is different, optical power spectrum for mobility detection and an intensity autocorrelation function for the DLS measurement, the partitioning and aggregation process is the same. The measurement process is then repeated n times, and the statistics of the n reported values are computed.

The problem is that for any given choice of parameters, some measurements will be well resolved, as characterized by a low relative standard deviation of replicates of the measurements, but others will not. In particular, small molecules require longer integrations or greater number of acquisitions than large particles to get the same statistical accuracy for both processes. However, the user does not know a priori whether an unknown sample will require long integrations or large number of acquisitions to achieve a desired level of accuracy. Current approaches for both DLS and ELS require a user to measure a sample, determine the resulting standard deviation of multiple measurements, and then adjust the collection parameters and retake data to optimize the measurement. Thus, there is a need for dynamically adjusting collection parameters such as the number of acquisitions and/or integration time for a given acquisition during the collection of raw acquisition data to automatically achieve a desired accuracy DLS is a ubiquitous and non-invasive measurement for the characterization of nano- and micro-scale particles in dispersion. ELS, on the other hand, is somewhat invasive due to the application of an electrical current to the particles that can damage the sample over time. Care must be taken to measure the sample with as low of an applied field and in a short a time as is practical. The adaptive algorithm is particularly useful to prevent excessively long collections that could result in sample degradation.

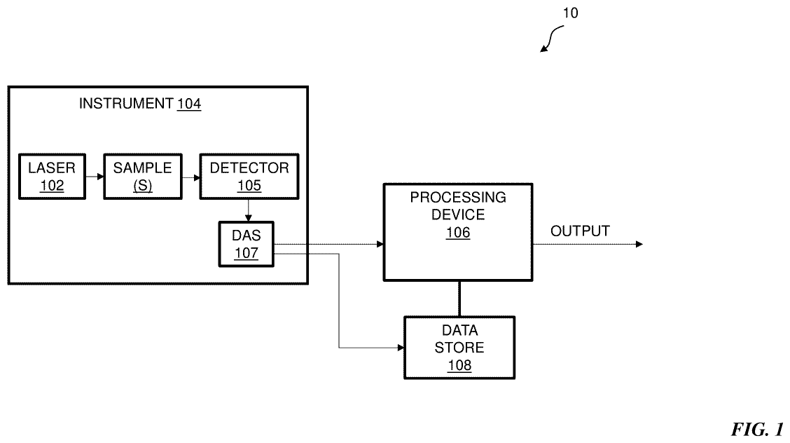

FIG. 1 depicts a block diagram of a system 10 at which embodiments of the present inventive concept can be practiced. The system 10 is constructed and arranged to perform dynamic light scattering (DLS) and electrophoretic mobility measurement operations, and may include an instrument 104 and a special-purpose processing device 106.

In some embodiments, the instrument 104 is constructed and arranged to measure electrophoretic mobility of particles dispersed in a liquid medium which implements dynamic light scattering (DLS) via directing light from a laser source 102 through a sample (S) of a dilute dispersion of particles in a liquid medium. Accordingly, the light scattering instrument can perform electrophoretic mobility and size measurements of proteins, polymers, and all types of nanoparticles. The sample (S) may be in a sample holder such as a plurality of wells or the like. The light source 102 may produce scattered light dependent on features of the particles in the sample, such as position, movement, velocity, and/or other features or characteristics of the particles of interest. The light source 102 may be a light emitting diode (LED) or a UV laser source or the like that directs a source of electromagnetic energy, e.g., a laser beam or other light, at a sample (S) at the instrument 104. The sample (S) may be held in a sample holder such as a plate, well, and so on. The source of light passes through the sample, irradiating the particles dispersed in it, so that light scattering caused by particles in the sample may occur. It is desirable for the system to perform measurements according to embodiments of the present inventive concept herein. In some embodiments, other measurements may be performed in the absence of a light source. For example, other electrophoretic mobility techniques include a power supply that applies an electric field to the sample (S) to measure particles.

The instrument 104 may include a detector 105 that detects the scattered light, radiation, conductivity, amperometry, and so on applied to the sample particles, and where resulting information is collected by a data acquisition system 107 which communicates with the detector 105. For example, samples are separated into analytes that flow in directions based on their inherent electrophoretic mobility and are then analyzed when they travel past the detector 105.

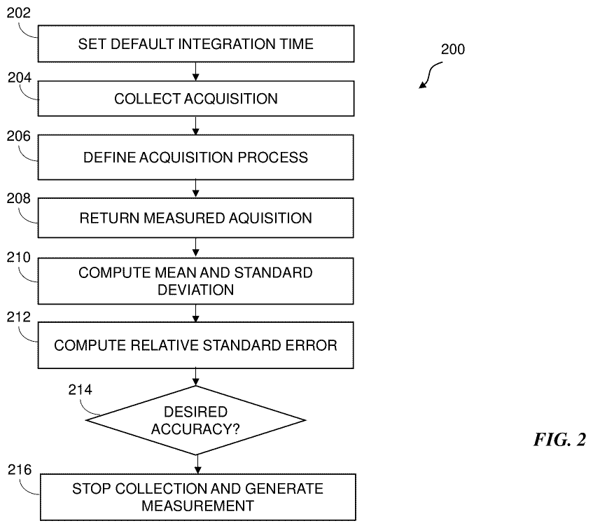

The instrument 104 outputs data collected by the data acquisition system 107 to the processing device 106, for example, data related to collected light scattered from the particles in the sample. The data is generated by the electrophoretic device 104 by collecting a plurality of acquisitions, which are used by the processing device 106 in addition to predetermined accuracy threshold data to determine measurement data. The processing device 106 can control the instrument by communicating with the data acquisition system 107 to cease collecting acquisition data when a sufficient number of acquisitions have been collected for generating a measurement, described in greater detail in FIG. 2 below.

In some embodiments, the system 10 includes a data store 108 that stores statistical accuracy threshold data, collection data, generated measurements, and/or other data related to adaptive time DLS and mobility collections, which can be used to determine the reproducibility of the measurements, each determined from a plurality of acquisitions. The data store 108 may include a database or other computer code for arranging the stored data for electronic retrieval. The data store 108 may include user inputs, for example, a keyboard and/or other computer peripheral devices for inputting data.

FIG. 2 depicts a flowchart of a computer implemented adaptive collection time method 200, in accordance with an embodiment. In some embodiments, the method 200 can be used for measuring the electrophoretic mobility of an unknown sample. In other embodiments, the method 200 can be used for light scattering measurements, for example, DLS measurements. In describing the method 200, reference is made to elements of FIG. 1. For example, for the computer implemented method, a system and computer program product can be configured to perform some, or all operations described in blocks 202-216 of the method 200.

At block 202, a default integration time is set. For example, a default integration time or length of time over which the detector output pulses are summed may be set for 1 second, but not limited thereto.

At block 204, an acquisition pertaining to a sample is collected. As described herein, a plurality of collected acquisitions can be aggregated into a single measurement. Each acquisition is integrated for the integration time established in block 202. The signals received from the detector 105 can be converted by the data acquisition system from analog to digital, and conveyed to a data port or the like for transmission to the computer processor 106 and/or data store 108 for storage and/or for further processing and analysis.

At block 206, a predetermined measurement process is applied to the acquisition. The measurement process may be any process known to one of ordinary skill in the art used to generate various results such as particle size or electrophoretic mobility. In some embodiments, the measurement process is defined according to equation (1), namely:

$${M}_{i} = M ( {A}_{i} ) \qquad ( 1 )$$

where Aᵢ is the acquisition integrated for the integration time. At block 308, depending on the instrument, for example, a DLS or electrophoretic mobility instrument, the function M(A) will return the measured signal, e.g., size, mobility, etc. via a predetermined process of fitting the acquisition data A to establish the repeatability and/or reproducibility of the generated measurements. The measurement process requires that the function M(A) returns a result having an accuracy of interest, and that is desirable to set.

At block 210, for each acquisition, in some embodiments the mean (equation (2) below) and standard deviation (equation (3) below) of the partial acquisition series is computed according to the following:

$${\overline{m}}_{i} = \frac{1}{i} {\sum }_{j = 0}^{i} {m}_{j} \qquad ( 2 )$$

$${σ}_{i} = \sqrt{\frac{1}{i} {\sum }_{j = 0}^{i} {( {m}_{j} - {\overline{m}}_{i} )}^{2}} \qquad ( 3 )$$

where i is the number of acquisitions that have been collected. At block 212, the relative standard error in the mean σₘ_{ᵢ} and the absolute error in the mean σₐ_{ᵢ} are computed, for example, according to equation (4) and (5):

$${σ}_{{m}_{i}} = {σ}_{i} / ( {\overline{m}}_{i} √ i ) \qquad ( 4 )$$

$${σ}_{{a}_{i}} = {σ}_{i} / √ i \qquad ( 5 )$$

Where σₘ_{ᵢ} and σₐ_{ᵢ} establishes the repeatability of the measurements taken for one or more acquisitions.

As the number of samples, or acquisitions (i), increases, the mean m̅ᵢ of the partial acquisition series will converge to the true underlying mean value and σ̅ᵢ will converge to the true standard deviation of the individual acquisitions, where the true values may be approximations derived from prior experimental data or theoretical predictions. Therefore, the relative standard error σₘ_{ᵢ} will decay towards zero as 1/√i. When this reaches a threshold desired accuracy t, for example, a determined threshold of 10%, then the system stops collecting data and concludes the measurement. This process can be repeated for the next measurement. Similarly, the absolute standard error σₐ_{ᵢ} will tend towards zero as 1/√i. When the mean value m̅ is very small, it will take a large number of acquisitions to cross the relative standard error threshold. In that case, it may be preferable to have an alternate threshold based on the absolute standard error σₐ_{ᵢ} crossing an absolute standard error threshold.

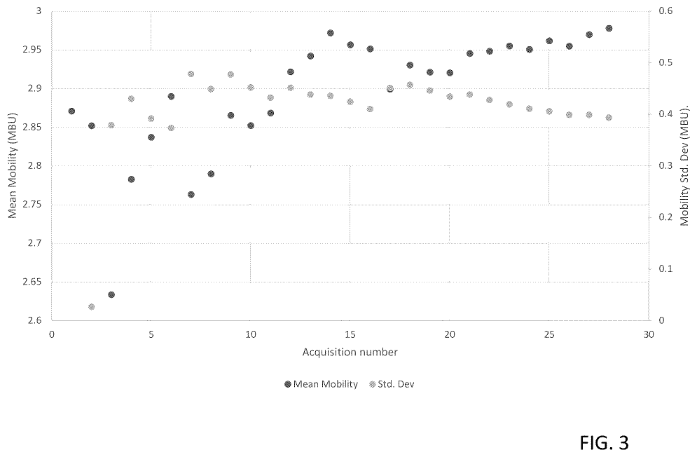

For example, FIG. 3 depicts an example of a mobility mean and standard deviation as a function of acquisition number (i). Both the mean mobility values and the standard deviation approach constant values as the number of acquisitions increases. Mobility is represented in units of mobility units (MBU) along the left axis (m̅ᵢ) which are in units of μm·cm (V sec). The mobility standard deviation values are along the right axis (σᵢ) which are also represented in MBU.

Referring again to the left axis (m̅ᵢ), the mean electrophoretic mobility is determined by computing the mobility on each acquisition (e.g., set for 1 second as provided in the example in block 202 of the method 200) and averaging all previous results. For example, the average mobility for the 25^{th} acquisition is constructed by determining an average value of the mobility for all 25 previous results. As shown in the left axis (mean value (m̅ᵢ)) FIG. 3, this converges to the true underlying sample mobility. On the right axis is the standard deviation of all previous acquisitions. It too converges on a fixed value, i.e., the standard deviation (σᵢ) converges to the true standard deviation of the individual 1 second acquisitions.

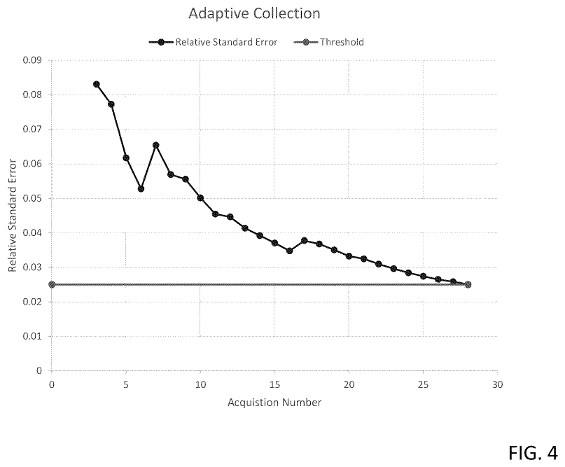

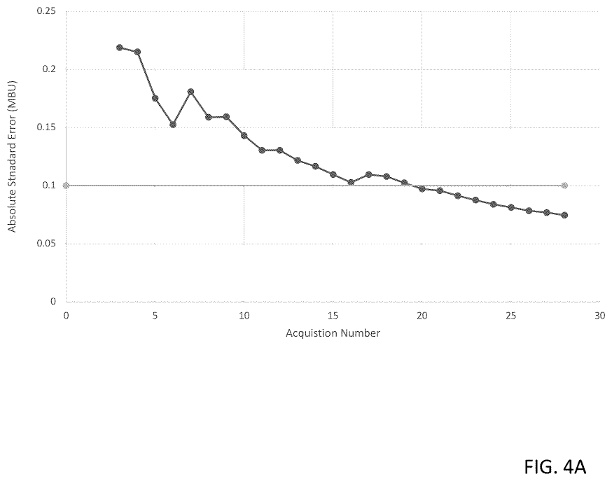

FIG. 4 depicts a graph illustrating a relationship between relative standard errors σₘ_{ᵢ} and the number of acquisitions i produced according to the adaptive collection time method 200 of FIG. 2. In particular, the graph illustrates a measurement of the relative standard error as the number of acquisitions increases. In this case the standard error threshold 302 is set to 2.5%. In accordance with block 212 of the method 200, the standard error 303 decreases as the number of acquisitions increases. When it passes the 2.5% threshold, the system stops collecting and the measurement is complete.

FIG. 4A shows what would happen if the stopping condition were applied to the absolute standard error σₐ_{ᵢ} with an absolute standard error threshold set to 0.1MBU. As shown on the y-axis the absolute standard error decreases as the acquisition increases until it passes the threshold of 0.1MBU. For this example, the system would have stopped at acquisition 20. The algorithm completes the measurement when either the relative standard error σₘ_{ᵢ} drops below the relative standard error threshold (2.5%) or when the absolute standard error σₐ_{ᵢ} drops below the absolute standard error threshold or the number of acquisitions exceeds the maximum number of acquisitions, whichever comes first.

If the noise associated with the short acquisitions follows a normal distribution, then the standard error of the mean computed from the acquisitions in a single measurement will be the same as standard deviation computed between multiple measurements. Another way of putting this is that if we choose the stopping condition for each measurement so that σₘ_{ᵢ}=2.5%, then when we compute the ordinary standard deviation of multiple measurements, it too should be 2.5%.

One problem that can occur is that the data fitting process M(A) may not be reliable when operating on individual acquisitions. This is a common issue when the analysis involves nonlinear least squares fitting of the acquisition data. When this occurs, some fits may yield correct, albeit noisy results, while others might yield spurious results due to a failed fitting process. This is not necessarily a problem since the partial standard errors of the acquisition series are only used to determine the criteria used by the system to stop the acquisition collection process for a given measurement so that the final measurement result can be generated. The actual analysis reported to the user is performed on the measurement data, which by construction has a much lower noise. However, the presence of spurious intermediate results can cause the acquisition sequence to take longer to cross the termination threshold than otherwise required. That means that the measurement data will have a lower overall error than the target. In order to avoid this issue, a predetermined threshold can be applied on the partial standard deviation sequence σᵢ. As the acquisition number i increases, the acquisition number i approaches a fixed value as defined above. If this value is greater than a user defined threshold, then the individual integration times can be increased. For the example above, the long term relative standard deviation

$$rsd = {lim}_{i \to ∞} {σ}_{i} / {\overline{m}}_{i} = 13.2 \% .$$

If this is too high for the function M(A) to return reliable results, the integration time can be increased adaptively. For Gaussian noise, σ∝1/√tᵢₙₜ, where tᵢₙₜ is the integration time, a new integration time can be set to be

$${t}_{new} = {{t}_{int} ( \frac{rsd}{{rsd}_{targ}} )}^{2} ,$$

where rsd_{targ} is the target relative standard deviation. In this case if a one second integration time per acquisition gave rsd=13.25% and the user desired value is rsd_{targ}=10%, then the integration time can be changed to

$$1 sec \times {( \frac{13.25}{10} )}^{2} = 1.8 sec .$$

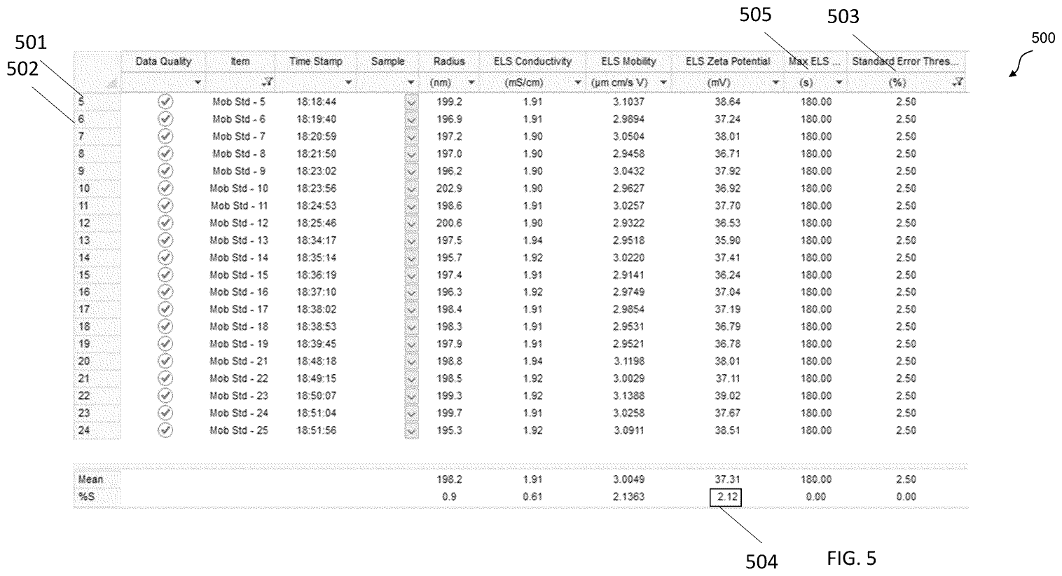

FIG. 5 shows an example of this concordance showing the results of 24 replicate measurements of a sample, each of which was collected using the adaptive algorithm. The figure shows a subset of the full data. For example, rows 501 and 502 are replicates. In this example the absolute standard error threshold was set to a zero (0) value, so all measurements completed by passing the relative standard error threshold of 2.5% as shown in column 503. The resulting replicate measurements had an ordinary standard deviation of 2.12% (see box 504). Column 505 displays an upper time limit of 180.00 seconds that is received by the system for the collecting (e.g., 5 minutes) that is allowed for each measurement. Since the algorithm is adaptive, each measurement will in general have a different number of acquisitions, but none will exceed the 180 second limit. As described above, the system collects data from an instrument until a relative standard error in a mean value (m̅ᵢ) (see equation (2) above) among an acquisition series of the data (e.g., i<N) crosses the accuracy threshold 402, i.e., 0.025 relative standard error in the mean in FIG. 4. In FIG. 5, the table illustrates that the replicate measurements had an ordinary standard deviation of 2.14% standard deviation. This is consistent with the goal that the reproducibility of the measurements is consistent with the relative standard error threshold set by the user.

As described above both the relative and absolute standard errors decay towards zero over a plurality of acquisitions until it reaches a threshold desired accuracy, then the system 10 stops collecting data and concludes the measurement. Since the partial standard deviations values σₘ_{ᵢ} and σₐ_{ᵢ} trend towards zero roughly as 1/√i, the system is guaranteed to cross the desired thresholds for a sufficiently large value of i, but the time required to complete a measurement might be excessive. This analysis assumes that the sample is physically stable, and the true underlying measured parameters do not change over time. However, is it possible that the sample degrades during the measurement, in which long collections times are undesirable. In this case, it is valuable to define absolute maximum N of the number of acquisitions to bound the collection time. Then the condition is to continuing collecting while σₘ_{ᵢ}>t and σₐ_{ᵢ}>t_{abs} and i<N.

In some embodiments, the speed and efficiency of the algorithm described with reference to the method 200 of FIG. 2 can be improved. Because each of the acquisitions contain a relatively small amount data, it is possible that they can give an inaccurate result. Alternatively, a sample may have a small number of impurities such as dust particles that are not representative of the sample being measured. In this case, some of the acquisitions can be culled from the final measurement. One simple method of performing this operation is when the algorithm has terminated, where any outlier measurements can be removed from the series. An outlier can be defined as any measurement that deviates from the mean by more than n σₘ_{ᵢ}, where n is a user defined parameter. For example, by setting n=3, then roughly 1% of the samples will be defined as outliers. In other words, any sample greater than 3σₘ_{ᵢ} can be discarded. The system can identify in the measurement the data that has not been culled.

The algorithm described with reference to the method 200 of FIG. 2 can be conducted in real time as the data are collected. However, the method 200 may be applied to a postprocessing version. Generally, the partitioning of the sequence of acquisitions into measurements is an arbitrary decision made by the user. As described in the problem statement, one can choose this partitioning to be a fixed number of acquisitions per measurement or by using the adaptive algorithm, one gets a constant standard error but each measurement will potentially contain a different number of acquisitions. These decisions are made by the user before the measurement is started. However, the system can collect all of the acquisitions for a series of measurements and then perform the partitioning a posteriori, so that the partitioning of acquisitions into measurements are performed only after all the acquisitions are collected. In the example above, one could then decide during the postprocessing to perform the partitioning to give 2.5% accurate data, or to choose a different threshold, for example, change the threshold to 5% accurate data. In the latter case, a larger number of measurements may be reported that have a larger standard deviation. This is useful to test the assumption that the sample is unchanging by intentionally choosing a higher threshold so that one can perform a trend analysis.

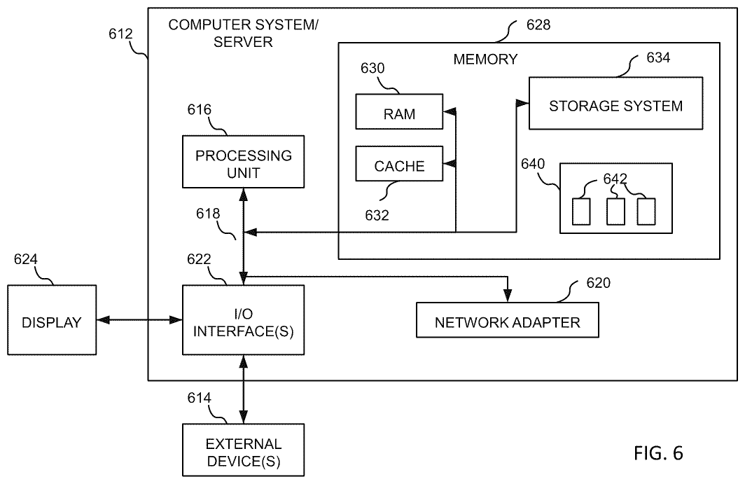

FIG. 6 depicts a computer system 600 in accordance with an exemplary embodiment. In an exemplary embodiment, the computer system 600 is a standalone computer system, a network of distributed computers, or a cloud computing node server. In some embodiments, the computer system 600 can perform some or all of the method 200 of FIG. 2. Computer system 600 is only one example of a computer system and is not intended to suggest any limitation as to the scope of use or functionality of embodiments of the present disclosure. Regardless, computer system 600 is capable of being implemented to perform and/or performing any of the functionality/operations of the present disclosure.

Computer system 600 includes a computer system/server 612, which is operational with numerous other general purpose or special purpose computing system environments or configurations. Examples of well-known computing systems, environments, and/or configurations that may be suitable for use with computer system/server 612 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices.

Computer system/server 612 may be described in the general context of computer system-executable instructions, such as program modules, being executed by a computer system. Generally, program modules may include routines, programs, objects, components, logic, and/or data structures that perform particular tasks or implement particular abstract data types. Computer system/server 612 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 612 in computer system 600 is shown in the form of a general-purpose computing device. The components of computer system/server 612 may include, but are not limited to, one or more processors or processing units 616, a system memory 628, and a bus 618 that couples various system components including system memory 628 to processor 616. The processor 616 can be similar to or the same as the computer processor 106 of FIG. 1.

Bus 618 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 612 typically includes a variety of computer system readable media. Such media may be any available media that is accessible by computer system/server 612, and includes both volatile and non-volatile media, removable and non-removable media.

System memory 628 can include computer system readable media in the form of volatile memory, such as random access memory (RAM) 630 and/or cache memory 632. The system memory 628 can include the data store 108 of FIG. 1.

Computer system/server 612 may further include other removable/non-removable, volatile/non-volatile computer system storage media. By way of example only, storage system 634 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 618 by one or more data media interfaces. As will be further depicted and described below, memory 628 may include at least one program product having a set (e.g., at least one) of program modules that are configured to carry out the functions/operations of embodiments of the disclosure.

Program/utility 640, having a set (at least one) of program modules 642, may be stored in memory 628 by way of example, and not limitation. Exemplary program modules 642 may include an operating system, one or more application programs, other program modules, and program data. Each of the operating system, one or more application programs, other program modules, and program data or some combination thereof, may include an implementation of a networking environment. Program modules 642 generally carry out the functions and/or methodologies of embodiments of the present disclosure.

Computer system/server 612 may also communicate with one or more external devices 614 such as a keyboard, a pointing device, a display 624, one or more devices that enable a user to interact with computer system/server 612, and/or any devices (e.g., network card, modem, etc.) that enable computer system/server 612 to communicate with one or more other computing devices. Such communication can occur via Input/Output (I/O) interfaces 622. Still yet, computer system/server 612 can communicate with one or more networks such as a local area network (LAN), a general wide area network (WAN), and/or a public network (e.g., the Internet) via network adapter 620. As depicted, network adapter 620 communicates with the other components of computer system/server 612 via bus 618. It should be understood that although not shown, other hardware and/or software components could be used in conjunction with computer system/server 612. Examples include, but are not limited to microcode, device drivers, redundant processing units, external disk drive arrays, RAID systems, tape drives, and data archival storage systems.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A method comprising:
receiving, from a data store by a computer system, a predetermined threshold related to a statistical accuracy for determining a measurement result related to a sample processed by an instrument;
collecting data regarding the sample from the instrument until a standard error of a mean among an acquisition series of the data is substantially equal to the predetermined threshold; and
generating the measurement result of the acquisition series of the data when the standard error is substantially equal to the predetermined threshold.

2. The method of claim 1, wherein collecting the data comprises defining each acquisition of the acquisition series on a measurement process as a function of the each acquisition integrated for an integration time.

3. The method of claim 2, wherein the measurement process generates a result including a particle size or electrophoretic mobility of the sample.

4. The method of claim 1 further comprising:
calculating, by the computer system, a mean value and a standard deviation value of an acquisition of the acquisition series;
calculating a relative standard error as a function of the mean value and the standard deviation value; and
determining that the relative standard error is the standard error when the relative standard error is substantially equal to the predetermined threshold.

5. The method of claim 4, as a number of acquisitions increases, the mean of the acquisition series converges to a true underlying mean value and the standard deviation of the acquisition series converges to a true standard deviation of individual acquisitions.

6. The method of claim 4 further comprising: controlling the instrument to cease providing additional acquisitions for the acquisition series in response to a determination that the relative standard error is substantially equal to the predetermined threshold.

7. The method of claim 1 further comprising:
providing a user-defined maximum time limit for the collective integration times of the acquisitions in the acquisition series.

8. The method of claim 1, wherein the instrument is an electrophoretic light scattering (ELS) measurement instrument and wherein the data collected from the instrument comprises electrophoretic mobility data from the ELS measurement instrument.

9. The method of claim 1, wherein the instrument is a dynamic light scattering (DLS) instrument for measuring diffusion behavior of molecules of the sample.

10. The method of claim 1, wherein the data regarding the sample is collected according to at least one collection parameter, including an integration time and a number of acquisitions for a desired sample measurement.

11. The method of claim 1, further comprising:
computing the standard error of the mean computed from the acquisitions in a single measurement that is the same as a standard deviation computed between multiple measurements.

12. The method of claim 1, further comprising after the data is collected, allowing for the choice of the threshold to use or the maximum number of acquisitions to use per measurement to be adjusted after-the-fact.

13. The method of claim 1, wherein the predetermined threshold comprises a relative standard error threshold or an absolute standard error threshold.

14. A method, comprising:
programming a special-purpose computer system with a set of collection parameters including a desired statistical accuracy and a maximum number of acquisitions;
collecting acquisition data for determining a measurement, the acquisition data collected according to the set of collection parameters;
as each subsequence acquisition is collected, computing a standard error of the mean of all acquisitions that have been completed, wherein when a value of a current acquisition crosses a predetermined threshold related to the statistical accuracy or the maximum number of acquisitions is reached, the system returns the measurement.

15. The method of claim 14, wherein collecting the acquisition data comprises defining each acquisition of an acquisition series on a measurement process as a function of the each acquisition integrated for an integration time.

16. The method of claim 14, wherein determining the measurement comprises generates a result including a particle size or electrophoretic mobility.

17. The method of claim 14, wherein the predetermined threshold comprises a relative standard error threshold or an absolute standard error threshold.

18. A computer program product comprising a computer readable storage medium having program instructions embodied therewith, the program instructions executable by a processor to cause the processor to perform a method comprising:
receiving a predetermined threshold related to a statistical accuracy for determining a measurement result related to a sample processed by an instrument;
collecting data regarding the sample from the instrument until a standard error in a mean among an acquisition series of the data is substantially equal to the predetermined threshold; and
generating the measurement result of the acquisition series of the data when the standard error is substantially equal to the predetermined threshold.

19. The computer program product of claim 18, wherein the predetermined threshold comprises a relative standard error threshold or an absolute standard error threshold.
